(19) United States
(12) Patent Application Publication
(10) Pub. No.: US 2008/0118460 A1
(43) Pub. Date: May 22, 2008

(54) Conjugates of polymer and pharmacologically active agents and a novel polymer blend

(76) Inventors: Evgeny Vulfson, Irvington, NY (US); Jeffrey W. Hare, Gillette, NJ (US)

Correspondence Address: Evgeny Vulfson, Apt 2, 36 Hamilton Road, Irvington, NY, 10533, US

(21) Appl. No.: 11/982,794

(22) Filed: Nov. 5, 2007

Related U.S. Application Data
(60) Provisional application No. 60/856,666, filed on Nov. 3, 2006.

(51) Int. Cl. A61K 48/00; A61K 39/395; C08G 63/91

(52) U.S. Cl. 424/78.27; 424/78.3; 525/54.1; 525/54.2

(57) ABSTRACT

The instant invention provides a novel conjugate of a synthetic, biodegradable, exceedingly hydrophilic and non-proteinaceous polymer, whereby each of the above attributes is defined in the specification, and various pharmacologically active agents. The instant invention also provides methods for the identification of said polymers, and methods for the preparation of conjugated pharmaceutically active agents. Furthermore, the invention provides a novel polymer blend for use in pharmaceutical and health care products and applications.

This application claims priority to the previously filed U.S. provisional application for patent No. 60/856,666, filed Nov. 3, 2006.

### FEDERALLY SPONSORED RESEARCH OR DEVELOPMENT

N/A

### PARTIES TO A JOINT RESEARCH AGREEMENT

N/A

### BACKGROUND OF THE INVENTION

1. Field of Invention

The present invention relates to polymer-pharmaceutically active agent conjugates and particularly, to small molecule drug- and protein-polymer conjugates, and a method of preparation thereof. This invention also relates to polymer blends and their use in pharmaceutical and health care products and applications.

2. Description of the Related Art

Protein-based biopharmaceuticals are expected to contribute greatly to the treatment of many human diseases. However, up to now only a relatively small number of protein therapeutics have received regulatory approval due to the well known difficulties in transforming pharmacologically active proteins into safe and efficacious drugs. For example, many such proteins undergo rapid degradation and/or clearance from the blood stream, and/or induce adverse immunological response.

Several technologies have been developed to address these issues and among them covalent chemical modification of target proteins with polyethylene glycol (herein after also referred to as polyethyleneoxide and PEG) and its derivatives, so-called PEGylation, is by far the most prominent. Indeed, a number of PEGylated proteins are currently available to patients e.g. Amgen's Neulasta® for the treatment of neutropenia, Schering-Plough's PEG-INTRON® and Roche's Pegasus® for the treatment of hepatitis C and several others.

It is generally accepted that several characteristics of PEG make it particularly suitable for pharmaceutical applications. For example, PEG is a hydrophilic polymer, i.e., it has high energy of hydration and, hence, PEG-modified proteins are highly soluble in body fluids. Also, PEG is a synthetic polymer and many of its properties such as, molecular weight, branching and end functionality can be easily modified and manipulated to minimize the detrimental effect of the chemical modification (attachment) on the pharmacological activity of the target. The latter is important because a substantial loss of biological activity has been reported for many PEG-modified proteins (Veronese F M, 2001) and, consequently, numerous modifications of the PEGylation technology have been developed to deal with this problem.

PEGylation has also been used for attaching small molecules drugs (see, for example, US Patent Application 20060182692 and U.S. Pat. No. 6,461,603, and references cited therein) to improve bioavailability and/or facilitate formulation, albeit to a much lesser extent. This is because a linear chain of PEG has only two functional groups at both ends which results in a highly unfavorable weigh ratio of the pharmaceutically active agent to polymer carrier. Although this can be somewhat improved by using branched PEGs (see, for example, U.S. Pat. No. 7,214,776, incorporated by reference herein), hydrophilic synthetic polymers with a higher density of functional groups for conjugating small molecular drugs, as disclosed, for example, in Hoste et al. (Hoste et al, 2004) are needed to overcome the shortcomings of PEGylation.

An improved solution to these important biomedical problems, as disclosed herein, is the use/application of an alternative polymer-protein conjugate, wherein the polymer component is (i) synthetic to facilitate the preparation of its structural analogs, subsequent chemical manipulation and targeting to specific functional groups on the protein surface, (ii) biodegradable to enable the release of the attached protein in a controlled (e.g. time dependent manner), for example, through the action of specific enzymes present in the serum or other body fluids, (iii) hydrophilic i.e. with the energy of hydration at least as high and preferably higher than that of PEG. It is an object of the present invention to provide such a polymer-protein conjugate and a method of preparation thereof. It is also an object of the present invention to provide a novel polymer-pharmaceutical agent conjugate, wherein multiple pharmaceutical agents are coupled to a single chain of synthetic, biodegradable and exceedingly hydrophilic polymer. Finally, it is an object of the present invention to provide a new polymer blend for use in pharmaceutical products and applications.

A variety of biodegradable polymers and their blends have been used for in medicine for a variety of purposes such as, for example, in drug formulation and controlled release, as a matrix for growing and regenerating cell and tissues, in medical implants and the like. Such polymers and their blends include both synthetic and natural polymers e.g. polyglycolic acid, polylactic acid and their co-polymers polyhydroxybutyrate, polyhydroxyvalerate and other polyhydroxyalkanoates and co-polymers thereof, and other polyesters, polycaprolactones, polydioxanone, polyanhydrides, polycyanoacrylates, various polysaccharides such as cellulose, starch, amylose, chitosan, alginate and derivatives thereof, and polypeptides such collagen, fibrinogen, fibrin and the like. The above mentioned polymers are used for drug delivery, guided tissue regeneration and tissue engineering, orthopedic applications, in medical stents and sutures, wound dressing, skeletal reconstruction, artificial skin (this list is given for the illustration purpose and is not intended to be limiting), typically in blends as described in great detail in “Biomaterial Science: An introduction to materials in medicine” (Rather B D et al., eds, 2004) and many other sources and textbooks well known to those skilled in the art.

According to these sources hydrophilicity of the blend is one of the defining characteristics of a biomedically useful polymeric blend, and the polymeric blend of a polymer or co-polymers of glyceric acid and its derivatives analogs and homologs with another comparably biodegradable polymer as disclosed in the present invention provides a higher overall hydrophilicity and a much widest range of hydrophilicities attainable in the final blend than any of the blends known in the prior art due to the exceedingly hydrophilic characteristic of the above said polymer or co-polymers of glyceric acid and its derivatives.

The use of PEG and its numerous derivatives for conjugating various pharmacologically active agents, including covalent modification of proteins, is well known in the prior art. However, PEG is not biodegradable, as defined hereinafter, and it is excreted from the human body in a largely unmodified form.

A number of other synthetic polymers e.g. polypropylene glycol (hereinafter after also referred to as polypropylene oxide and PPO), various acrylates and methacrylates, and vinyl pyrrolidones have been used for covalent modification of proteins and preparation of various polymer-drug conjugates. However, none of these polymers are biodegradable as defined herein and most of them are less hydrophilic than PEG, i.e., they have lower hydration energy.

A number of biodegradable polymers e.g. polyglycolic acid (PGA), polylactic acid (PLA) and some other polyhydroxy acids (PHAs) have also been used for covalent modification of proteins and preparation of various polymer-drug conjugates. However, the hydration energy of these polymers is typically lower than that of PEG and none are exceedingly hydrophilic, as defined hereinafter.

A number of naturally occurring polymers and their derivatives, e.g., proteins such as albumin and ferritin, antibodies and their fragments, and polysaccharides such as dextrans, polymers of sialic acids, and their derivatives, and the like have been used for covalent modification of proteins and preparation of various polymer-drug conjugates, but none of these polymers are synthetic, as defined hereinafter.

A number of polypeptides and their derivatives, e.g., poly-L-glutamic acid, poly-L-lysine, and other polymers of natural amino acids and their derivatives have been used for covalent modification of proteins and preparation of polymer-drug conjugates. However, all these polymers are “proteinaceous”, as defined hereinafter, and, therefore, their conjugates differ from the conjugates of the present invention.

A number of polymers containing about 1 mole of hydroxyl group per mole of monomer, e.g., hydroxypropylmethacrylamide, polyvinyl alcohol, and hydroxy-alkyl derivatives of various proteinaceous polymers such as, for example, poly(N-(2-hydroxyethyl-L-glutamine) have been used for covalent modification of proteins and preparation of polymer-drug conjugates, but none of these polymers combine all of the desirable characteristics of a polymer for the purposes of the present invention, as disclosed and described above: synthetic, biodegradable, exceedingly hydrophilic and non-proteinaceous. The combination of all four of these characteristics, each as defined hereinafter, are the distinct and distinguishing features of the polymers and polymer conjugates of the present invention. The differences between the conjugates of the present invention and those known in the prior art are summarized in Table 1, below.

TABLE 1
Polymers used for the conjugation
of pharmaceutically active agents

| | Biode- | | | Protein- |
|---|---|---|---|---|
| Polymer | gradable | Hydrophilic* | Synthetic | aceous |
| PEG | No | No | Yes | No |
| PPO | No | No | Yes | No |
| (Meth)acrylates | No | No | Yes | No |
| Vinyl pyrrolidones | No | No | Yes | No |
| Polylactate | Yes | No | No | No |
| Polyglycolic acid | Yes | No | Yes | No |
| PHAs | Yes | No | Varied | No |
| Polysaccharides and | Yes | n/d | No | No |
| their derivatives | | | | |
| Polypeptides and their | Yes | n/d | No | Yes |
| derivatives | | | | |
| Polymer of the present | Yes | Yes | Yes | No |
| invention | | | | |

*defined as a polymer with hydration energy appreciably higher than that of PEG.

### SUMMARY OF THE INVENTION

The current invention relates to conjugates of (a) synthetic, (b) biodegradable, (c) non-proteinaceous, and (d) exceedingly hydrophilic polymers, all as defined herein after, that hence have significant advantages over those of the prior art, and pharmacologically active agents. In a preferred embodiment, the above polymer-pharmaceutical agent conjugates contain a substantial proportion of glyceric acid, or analogs, homologs or derivatives thereof. In another preferred embodiment, the above polymers are prepared with the aid of biological catalysts, such as enzymes or catalytic nucleic acids.

The agent/substance with known pharmacological activity can be a low molecular weight monomeric substance (hereinafter “small molecule”) or a polymeric substance, for example, a oligo-, polypeptide, or protein, including and derivatives and analogs thereof, or a nucleic acid polymer, or a peptidomimetic.

The present invention also provides a method for the preparation of conjugated pharmaceutically active agents, whereby the method comprises:
1. Synthesis of a biodegradable polymer or co-polymer of the invention, such as, for example, a polymer or co-polymer of glyceric acid and its analogs, homologs and derivatives with an average molecular weight from about 1 kD to about 100 kD;
2. Producing or otherwise obtaining the pharmacologically active agent of the invention;
3. Covalently coupling of thus obtained polymer to the substance with known pharmacological activity; and
4. Purifying the resulting pharmaceutically active agent from unreacted materials and side-products.

Furthermore, the invention relates to conjugates of the above polymers and small molecules with pharmacological activity and therapeutic potential.

Finally, the invention relates to blends of two or more polymers, whereby the first polymer is a polymer or co-polymer of glyceric acid, or a derivative, homolog, or analog thereof, and (b) said first polymer and at least one of the other polymers present in the blend have comparable biodegradability, and wherein (c) the mass ratio of the first polymer to the rest of the polymers in the blend varies between 97:3 to about 3:97. In a preferred embodiment, the first polymer is exceedingly hydrophilic with a water hydration factor of 0.50 or higher.

### BRIEF DESCRIPTION OF DRAWINGS

FIG. 1. The conformations of polymers mentioned in the body of the application as obtained in MD calculations are shown. Carboxy-PEG: (a)—flat conformation and (b)—coil conformation, carboxyPPO: (c)—R flat and (d)—RS coil, (e)—acm-iso coil, (f)—pvp-iso flat, (g)—hpma-syn flat (h)—hpma-iso coil, (i)—glycolate coil, (j)—lactate RS flat, (k)—glycerateR flat and (l)—glycerateRS coil. See Detailed Description of the Invention and Example sections of the disclosure.

### DETAILED DESCRIPTION OF THE INVENTION

The invention described herein comprises conjugates of pharmacologically active agents with a known pharmacological activity and/or effect, as defined hereinafter, and one or more (a) synthetic, (b) biodegradable, (c) non-proteinaceous, and (d) exceedingly hydrophilic polymers, as defined hereinafter. Furthermore, the invention comprises the method of assaying for, and identifying such polymers, and the method of conjugating such polymers to said pharmacologically active agents. Finally the invention comprises blends of polymers, as defined hereinafter, said blends having utility for the formulation of pharmacologically active agents, as described below.

### Polymers of the Invention

For the purpose of this invention the term “polymer” is understood to mean a chemical entity comprising multiple repeating units (“monomers”) of the same (“homopolymers”) or different (“co-polymers”) chemical structures covalently linked to each other, wherein the number of monomers in the polymer is no less than five and the molecular weight of the resulting assembly is no less than about 500.

For the purpose of this invention the term “naturally-occurring” polymer is understood to mean any polymer that is present in living cells or can be synthesized by living cells without extensive genetic manipulation, including any further chemical alterations or modifications of the said polymer. For example, according to this invention poly-y-L-glutamate, many poly-saccharides and poly-peptides, including derivatives thereof, are naturally-occurring polymers. Conversely, “synthetic” polymers are not normally found or synthesized in living cells. For example, PEG and various poly-(meth)acrylates are, in accordance with this invention, synthetic polymers. It is explicitly understood that naturally-occurring polymers can also be synthesized in the laboratory, if necessary or desirable, and that synthetic polymers can, in principle, be produced in genetically engineered cells, provided that the cells are subjected to extensive genetic manipulation such as, for example, insertion of foreign gene(s) that are not naturally present in the said cells.

For the purpose of this invention the term “biodegradable” polymer is understood to mean a polymer that can be substantially degraded in the body. For example, the majority of naturally occurring polymers such as, poly-peptides, poly-saccharides and poly-esters can be cleaved by various proteases, glycosidases and esterases respectively; hence in accordance to this invention these polymers are biodegradable. Conversely, polymers such as PEG and many poly-(meth)acrylates are not biodegradable because these polymers are not substantially degraded or otherwise modified in the body and are typically excreted in substantially unchanged form. In accordance with this invention two polymers have “comparable” biodegradability, if they are degraded in the body at similar rates e.g. the difference in the rate constants of degradation between the two polymers is not much higher than about one order of magnitude.

For the purpose of this invention the term “proteinaceous” polymer is understood to mean any polymer which backbone comprises primarily the 20 natural L-amino acids commonly present in polypeptides and proteins synthesized by living organisms, including any further chemical or biological alterations or modifications. In accordance with this invention, any polymer comprising any number of natural L-amino acids combined in any order, chemically modified or not, is a “proteinaceous” polymer. Conversely, a polymer containing a substantial number of monomers other than the common 20 natural L-amino acids in its backbone is a “non-proteinaceous” polymer. For example, according to this invention poly(N-(2-hydroxyethyl-L-glutamine) and glycosylated poly-L-lysine are proteinaceous polymers because their backbones are built from L-glutamine and L-lysine respectively and further modified to give the corresponding hydroxyl-alkyl derivatives, while the same polymers containing D-glutamine and D-lysine would be “non-proteinaceous” polymers.

For the purpose of this invention the term “exceedingly hydrophilic” polymer is understood to mean a polymer with the hydration energy per surface area (SAHE) appreciably exceeding that of PEG, which is commonly perceived as a hydrophilic polymer. As exemplified herein, PEG has a water hydration factor (WHF) of 0.36, while the polymers of the conjugate of the present invention have WHF of about 0.50 or higher and, hence, according to the present invention, are exceedingly hydrophilic (see Example Section).

Other synthetic polymers used or proposed for use in polymer-pharmaceutical agent conjugates are about as hydrophilic as PEG but none of these are exceedingly hydrophilic as defined herein. Furthermore, none of the above polymers are biodegradable as defined herein.

Other biodegradable non-proteinaceous polymers previously used in polymer-drug conjugates, such as several polyesters, have also been analyzed with the aim of determining their WHF. Firstly, a number of polyesters from highly water soluble monomers were constructed, computed and compared. An approach analogous to that taken with the polyethers was chosen for assembling chain conformations. The extended conformation and at least one coil conformation were generated to determine limiting hydration energies for these systems. Extensive molecular dynamics with the chain geometry allowed to move are expected to lie between these limits as was the case for polyethers.

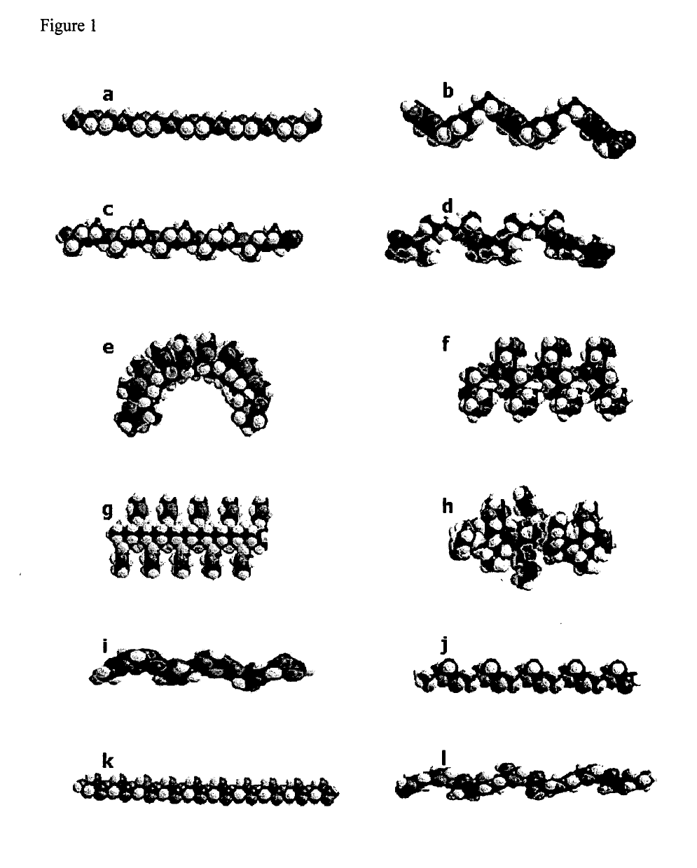

The simplest is polyglycolate, the polymer of 2-hydroxy-acetic acid, or glycolic acid. The coil geometry is generated by twisting the carbon-carbon backbone torsion angle to 60° analogous to the coil conformer of PEG and PPO. It has only a slightly more favorable interaction with water than the extended conformer (FIG. 1i). The WHF of polyglycolate lies somewhat higher than that of the soluble conformer of PEG, due to the backbone ester group. Adding a methyl group to the backbone carbon of glycolic acid yields 2-hydroxy-propanoic acid, commonly known as lactic acid. This naturally occurring acid is optically active, so both the isotactic poly-R-lactate and the syndiotactic poly-RS-lactate were constructed. As observed for PEG, adding the hydrophobic methyl group to polyglycolate lowers the WHF of the resulting polymers by about 3-5%. Interestingly, polylactates are unusual in that the extended form is more hydrophilic than the coil conformer (FIG. 1j). Placing this extra carbon in the polymer backbone has an even more negative affect on the WHF. The WHF of polyester formed from 3-hydroxy-propanoic acid, or β-lactic acid lies 3-4% below polylactates.

These simple polyesters are remarkably like their polyether counterparts. Polyglycolate having a value of WHF slightly higher than that of PEG and the polylactates being just above the general range occupied by PPO. Since addition of a methylene group to the polymer backbone has a negative effect on the hydration energy, any homo-polymers of higher monohydroxy alconoic acids would be insufficiently hydrophilic for use in the polymer-pharmaceutical agent conjugates of the present invention, despite the fact that these polymers are or can be synthetic, biodegradable and non-proteinaceous. It is also evident from the above that the polymer-conjugate as disclosed in the present invention is not known in the prior art.

In one of the preferred embodiments the polymer component of the conjugate disclosed herein is a polymer or co-polymer containing from 0.2 to 1.8 moles of hydroxyl groups per mole of monomer, and preferably from 0.6 to 1.4 moles of hydroxyl groups per mole monomer. Examples of such polymers include polymers and co-polymers of glyceric acid and its derivatives, homologs and analogs, preferably of a single enantiomer of glyceric acid and its derivatives, homologs and analogs. The number of hydroxyl groups in such polymers can be easily controlled and adjusted by co-polymerizing glyceric acid with monomers containing fewer hydroxyls, for example, lactic acid or modifying the resulting polymer with substances containing more hydroxyls, for example, monosaccharides. All such polymers and their modifications and derivatives can be used successfully to practice the present invention. As defined herein, such polymers are non-proteinaceous as its backbone does not consist primarily from the 20 natural amino acids, synthetic as they do not occur in living cells, biodegradable as they can be degraded in the body e.g. by esterases ubiquitously present in the serum and other body fluids, and exceedingly hydrophilic as exemplified by molecular dynamic calculations presented herein.

The polymer component of the conjugate disclosed herein can be a linear, or substantially linear or a branched polymer. For the purpose of this invention the term “substantially linear” is understood to mean a polymer containing no more than about one branching point per about 300 monomer units. Branched polymers can be randomly branched polymers or polymers with controlled shape, form or morphology, including but not limited to star, comb, dendrimeric polymers and the like.

The polymers of the present invention may also be covalently linked or otherwise attached to other known polymers, for example in the form of block polymers, grafts or any other forms known to one of ordinary skill in the art. The resulting polymer is substantially synthetic, biodegradable, non-proteinaceous and exceedingly hydrophilic.

After extensive study and research (see Example), some non-proteinaceous, synthetic, biodegradable, and exceedingly hydrophilic polymers have been found and identified. For example, such a polymer can be made from 2,3 dihydroxy-propanoic acid or glyceric acid to give a non-proteinaceous, synthetic and biodegradable polyester. Glyceric acid is chiral, so both an isotactic R polymer and a syndiotactic RS polymer were generated. In the first example the β-carbon was used as the polymerization site, so these polymers are referred to as β-glycerates. With multiple atoms in the polymer backbone there are multiple coil conformers that can be constructed (FIG. 1 k,l). One representative conformer is listed in the table for each of the two glycerate chains. As evident from the Table below, the coil structures have very high WHF values of above 0.5 and even the flat conformers have WHF values of about 0.5 or higher, i.e., these polymers are appreciably more hydrophilic than PEG. Glyceric acid can also be polymerized using the hydroxyl function of the α-carbon. The corresponding conformations of α-glycerates were also tested and proved to be equally hydrophilic. One would expect that polymers containing mixtures of the α- and β-polymers would also be “exceedingly hydrophilic”. Thus, one can conclude that polyglycerates, and generally polymers rich in glyceric acid and its analogs, homologs and derivatives, are exceedingly hydrophilic polymers as defined herein.

### Synthesis of Polymers of the Invention

The polymer synthesis (step 1) can be carried using conventional methods of polymerization known to one of ordinary skill in the art, such as, for example, but not limited to, as disclosed in detail in “Polymer Synthesis: Theory and Practice: Fundamentals, Methods, Experiments” (Braun D, 2002) and “Handbook of Polymer Reaction Engineering” (Meyer and Keurentjes, eds, 2005). For example, polymers containing glyceric acid, such as, for example, polyesters, can be prepared in accordance to the above manuals, which describes a variety of methods, techniques and catalysts for making low and high molecular weight polymers, linear and branched, using monomers such as alcohols, diols, triols and carboxylic acids and hydroxyl-carboxylic acids and their derivatives. Other methods and techniques of polymer synthesis known to one of ordinary skill in the art, for example, as described in the above references, for the synthesis of other polymers such as, for example, but not limited to polyesters, polyether, polyamides, can also be employed successfully for preparing polymers of the present invention.

It is preferable to use such methods that enable the preparation of substantially linear polymers as defined herein, preferably such method that, for example, minimize racemization of enantiomerically pure glyceric acid and its analogs, homologs and derivatives. For example, one of the preferred methods for polymerizing enantiomerically pure monomers is by means of biocatalysis, preferably using enzymes such as commercially available enzymes, e.g. esterases from Novozyme (Denmark), Genencor (USA) and other suppliers, using alkyl esters or otherwise activated e.g. vinyl esters and the like of glyceric acid or its analogs, homologs and derivatives, preferably under conditions that favor the equilibrium shift towards polymerization, for example, in non-aqueous solvents and/or under vacuum, which may be applied continuously or from time to time as necessary or desirable. A variety of enzymes, methods and techniques for carrying out such reaction are described in “Methods in Biotechnology: Enzymes in Nonaqueous Solvents” (Vulfson et al, eds, 2001), and references cited therein. Those skilled in the art would instantly recognize that other methods suitable for the preparation of such polymers are also known and can be used to successfully practice the present invention.

Once obtained, the polymer is precipitated from the reaction by, for example, the addition of a non-solvent, dried and subjected to conventional analysis to confirm the degree of polymerization and polydispersity. A number of such methods are provided in the manuals cited above, for example GPC, light scattering, mass spectrometry and the like. If desired, the degree of polymerization can be followed by using the same methods by withdrawing an aliquot from the polymerization mixture and the reaction is preferably stopped when the polymer of the target molecular weight e.g. from 1 kD to 100 kD is formed.

### Assaying Polymers of the Invention

Polymers of the invention are non-proteinaceous, synthetic, biodegradable, and exceedingly hydrophilic, each as described and defined in detail above. These attributes may be assayed, individually or in combination, by any method or methods know to one of ordinary skill in the art.

As a non-limiting example, the biodegradable attribute of a polymer may be assayed by adding it to serum, incubating for a period of time, or several periods of time (e.g. time-course experiments), and assaying for breakdown products, such as mono- and oligomers. Such assays may, as a non-limiting example, include mass-spectrometric analysis of samples to determine the decrease in molecular weight of the polymer due to its biodegradation by, for example, enzymes present in the plasma.

Furthermore, the energy of hydration for polymers disclosed herein can be experimentally determined by any method known to one of ordinary skill in the art, including, but not limit limited to, calorimetry. For example, a commercially available calorimeter, such as, for example, but not limited to, the TA Instruments Precision Solution Calorimeter (TA Instruments, 109 Lukens Dr., New Castle, Del. 19720) or the Parr model 6755 Solution Calorimeter (Parr Instrument Company, 211 Fifty Third Street, Moline, Ill. 61265) can all be used successfully for performing such measurement in accordance to instruction manuals provided by the above manufacturers. Hydrophilicity may also be determined by any computational methods known to one of ordinary skill in the art, such as, for example, the methods described in the Examples hereinafter.

### Pharmacologically Active Agents of the Invention

The pharmacologically active agents of the invention are limited to those agents with at least one known pharmacological activity with potential utility for the treatment of at least one condition, disorder, or disease. Such pharmacologically active agents of the invention include, but are not limited to, pharmacologically active monomeric small molecules, whether synthetic or derived, purified, or otherwise obtained from natural or non-natural sources, oligopeptides, polypeptides, proteins, protein complexes, catalytic polypeptides, enzymes, cytokines, binding proteins, antibodies, and fragments, derivatives, and analogs of any of the above, peptidomimetics, and other biologically or synthetically derived molecules, such as, for example, but not limited to, nucleic acid molecules, including, but not limited to, RNA molecules, such as, for example, aptamers and RNAi molecules, and DNA molecules, such as, for example vectors and inserts of therapeutic sequences.

### Oligo- and Polypeptides and Peptidomimetics of the Invention

Oligo- and polypeptides, proteins, and protein complexes of the invention comprise oligo- and polypeptides that remain functionally active upon application of the instant invention. The polypeptides of the invention are limited to those with at least one known pharmacological activity with potential utility for the treatment of at least one condition, disorder, or disease. Nucleic acids encoding the foregoing polypeptides are also provided. The term “functionally active” material, as used herein, refers to that material displaying one or more functional activities or functionalities associated with one or more of the oligo- or polypeptides, or of a polypeptide complex. Such activities or functionalities may be the oligo- or polypeptide's, or polypeptide complexes' original, natural or wild type activities or functionalities, or otherwise. Also included within the scope of the pharmacological agents or compostions of the invention are: fragments; fusions, comprising one or several of the polypeptides; mutants, including, but not limited to, point mutations, whether or not conservative, insertions, and deletions; derivatives; analogs of oligo- and polypeptides, proteins, or protein complexes, and furthermore post-translationally or chemically modified, naturally or recombinantly produced and chemically synthesized oligo- and polypeptides, proteins, or protein complexes comprising only naturally occurring amino acids, only non-naturally occurring amino acids, and both naturally and non-naturally occurring amino acids, each as disclosed in detail in Marshall et al. (U.S. Pat. No. 7,037,894), which is hereby incorporated herein by reference.

Any method known to one of ordinary skill in the art may be used to obtain an oligo- or polypeptide, or polypeptide complex of the invention to be conjugated and/or formulated according to the methods of the invention. An oligo- or polypeptide or polypeptide complex of the inventions may be obtained, for example, by any protein purification method known in the art from any natural or non-natural source, including, but not limited to, organisms, tissues, samples, and cell-lines that either naturally, recombinantly, or otherwise express the oligo- or polypeptide, or polypeptide complex of the invention. All sources and methods of obtaining polypeptides described in Marshall et al. (U.S. Pat. No. 7,037,894) are included in the scope of this invention, and the reference is incorporated by reference, as disclosed above.

Furthermore, peptidomimetics, such as, for example, but not limited to, described in detail in “Peptidomimetics Protocols: Methods in Molecular Medicine” (Kazmierski, ed, 1998) are within the scope of the pharmaceutically active agents, compounds, and compositions of the invention.

### Small Molecule Drugs

Small molecule drugs of the invention, including those that are covalently conjugated to polymers of the invention and those formulated in polymer blends of the invention, either according to the methods described herein, are pharmacologically active monomeric small molecules, whether synthetic or derived, purified, or otherwise obtained from natural sources. Small molecules of the invention are limited to those with at least one known pharmacological activity with potential utility for the treatment of at least one condition, disorder, or disease. Such pharmacological activity may have been identified by any means known to one of ordinary skill in the art, including, for example, high through-put and high content screening assays, including, but not limited to, such methods as described in detail in such references as Proll et al., 2007; Krausz 2007; Perrimon & Mathey-Prevot, 2007; Luesch, 2006; Douris et al., 2006; Krausz, 2007; Chen, 2006; Rausch, 2006; Leifert, 2005; Rausch, 2005; Blake et al., 2007; Vogt et al., 2004; Zanella et al., 2007; Lundholt, 2006; Trask et al., 2006; Jose, 2006; Eglen, 2005; Hamdan et al., 2005.

Small molecules may be isolated, derived, purified, or otherwise obtained from natural or non-natural sources by any method(s) know to one of ordinary skill in the art, or may be synthesized by any method(s), including such methods as described in “Wilson & Gisvold's Textbook of Organic Medicinal and Pharmaceutical Chemistry” (Block & Beale, 2003), or any other method(s) known to one of ordinary skill in the art.

### Nucleic Acid Polymers of the Invention

Nucleic acids of the invention include RNA and DNA monomers, and any analogs or derivatives thereof. RNA molecules, include, but are not limited to, antisense, siRNA, RNAi, aptomers, and any other RNA molecules, whether regulatory, having binding activity, or catalytic activity, as described, both with regard to their definitions and with regard to methods of manufacturing, for example, but not limited to, in U.S. Pat. No. 6,852,535, U.S. Pat. No. 6,653,458, U.S. Pat. No. 6,387,617, or any of the references listed in any of the above US patent applications, which are each incorporated in their entirety by reference herein, are included in the scope of the present invention.

### Conjugates of the Invention

The conjugate of the present invention comprises about one molecule of the pharmaceutical agent coupled to about one polymer chain or multiple pharmaceutical agents (which can be the same or different chemical entities) coupled to a single polymer chain, depending on the properties of the polymer, pharmaceutical agent and the desire of the practitioner. For example, for polymer-pharmaceutical agent conjugates such as conjugates of therapeutic polypeptides and proteins, it is preferable to have about one molecule of such agent coupled to about one molecule of the polymer. Conversely, for non-polymeric pharmaceutical agents the coupling of multiple molecules of the said agent to a single polymer is preferred. Such coupling of pharmaceutical agents to polymers is known to improve their therapeutic properties such as, for example, therapeutic index and pharmokinetics. It is explicitly understood in the present invention that multiple molecules of pharmaceutical agent can be molecularly the same or different chemical entities.

The polymer and the pharmaceutical agent in the conjugate of the present invention can be coupled directly to each other or through a specially designed linker. According to this invention, a linker can be any chemical entity capable of forming a covalent bond between the polymer and the pharmaceutical agent of the conjugate disclosed herein. A large number of such linkers are known in the prior art and all of them can be used to successfully practice the present invention (see, for example, “Bioconjugate Techniques”, Hermanson, G T, 1996; “Bioconjugation Protocols: Strategies and Methods”, Niemeyer, ed, 2004; “Chemical Reagents for Protein Modification”, Lundblad R L, 2004; “Polymer Synthesis: Theory and Practice: Fundamentals, Methods, Experiments”, Braun et al., 2002; and “Handbook of Polymer Reaction Engineering, Meyer and Keurentjes, eds, 2005).

The conjugate of the present invention is preferably soluble at pharmacologically active concentration in a largely aqueous medium and at about neutral pH, preferably at about 10 times the pharmacologically active concentration, preferably at about 100 times.

The present invention also provides a method for the preparation of pharmaceutically active agents; the method comprising (1) synthesis of biodegradable polymer or co-polymer of glyceric acid and its analogs, homologs and derivatives with an average molecular weight from about 1 kD to about 100 kD; (2) covalently coupling thus obtained polymer to a substance with known pharmacological activity and (3) purifying the resulting pharmaceutically active agent from unreacted materials and side-products.

### Attachment of Polymers

A variety of methods, reagents and techniques are well known for coupling polymers to pharmacologically active substances, including polymers and polypeptides (Hermanson, GT “Bioconjugate Techniques”. Academic Press, 1996; “Bioconjugation Protocols: Strategies and Methods” (Niemeyer, ed). CM Humana Press, 2004; Lundblad R L “Chemical Reagents for Protein Modification” CRC, 2004, Hoste, 2004). Typically, such coupling involves reacting a functional group on the polymer with a functional group of the pharmacologically active substance either directly or through a linker as described in the above cited manuals. For example, a polymer containing glyceric acid can be coupled to a protein by reacting a carboxyl group on the above said polymer, for example the terminal carboxyl, with an amino group on, for example, polypeptide or protein using a carbodiimide such as N,N′-dicyclohexylcarbodiimide and the like in accordance to the above cited manuals. Such coupling may also be accomplished in a two step reaction whereby a link of the desired length is first coupled to either the polymer or the pharmacologically active substance and the two are then covalently linked to each other. A variety of chemical groups such as for example amino-carboxyl-hydroxyl-thiol-groups, phenols and aldehydes, depending on the functionality of the polymer, pharmacologically active substance and the linker, can all be successfully linked using a variety of methods and reagents, in water, water-organic solvents mixtures or in organic solvents, described in the above manuals and using other methods well known to those skilled in the art. Once linked, the polymer-conjugate of the present invention can be separated from the mixture by a variety of methods, for example, but not limited to, chromatographic techniques, and characterized, for example, but not limited to, by mass-spectrometry.

The agent/substance with known pharmacological activity is preferably selected from the group comprising polypeptides, proteins and their derivatives and analogs, and the coupling of the polymer and the substance with known pharmacological activity to obtain the conjugate of the present invention is preferably carried out in water or polar organic solvents, or a mixture thereof. The above said mixture is preferably selected in such a manner as to enable sufficient reactivity between the polymer and the pharmacologically active substance and, at the same time, to avoid any damage or decomposition or any other alteration that might affect the said agent/substance, such as, for example, denaturation or oxidation of a protein. Many such mixtures and conditions are well known to skillful artisans.

The above said coupling can be accomplished directly or, optionally, through a linker. Depending on the chemical structures of the polymers and pharmaceutically active agents of the invention and on the number of attachment sites on each thereof, a single or multiple small molecule pharmaceutically active agents of the invention may be coupled to a single polymer of the invention, and, vice versa, a single or multiple polymers of the invention may be attached to a single pharmaceutically active agent of the invention. The resulting pharmaceutically active agent can be purified from, for example, unreacted materials and side-products. Any purification method known to one of ordinary skill in the art may be applied.

### Polymer Blends of the Invention

This invention provides, furthermore, a blend of at least two polymers, wherein (a) the first polymer is a synthetic biodegradable polymer or co-polymers of glyceric acid and its derivatives, analogs and homologs and (b) at least one of other polymers present in the blend has biodegradability comparable to that of the first polymer and (c) the mass ratio of the first polymer to all rest of the polymers in the blend varies between about 97:3 to about 3:97, preferably from about 9:1 to about 1:9.

The first polymer in the said blend is preferably an exceedingly hydrophilic polymer, preferably a substantial linear polymer, preferably with constituent glyceric acid and its derivatives, homologs and analogs being single enantiomers.

Any other comparably biodegradable polymer, naturally occurring or synthetic, can be used in the blend, e.g., various polyesters such as polylactic, polyglycolic and other (poly)hydroxy-acids and/or various polyamides, and the like. Such blends are particularly useful for encapsulation of drugs and other pharmacologically active substances, e.g., for the purpose of controlling their release, and can be used in many other health care products and applications.

Blending of the polymers disclosed herein for pharmaceutical purposes, such as, for example, drug formulation and controlled release, or, as another non-limiting example, for use as a matrix for growing and regenerating cell and tissues or medical implants (see Background of the Invention), can be accomplished by a variety of methods known to one of ordinary skill in the art. As a non-limiting example, polymers can be melted and combined or dissolved in the common solvent and dried as is routine practiced in the pharmaceutical industry.

### Formulation of Conjugates of the Invention

Pharmacologically active agents that are conjugated to polymers of the present invention are formulated according to methods known to one of ordinary skill in the art, including, for example, but not limited to, according to the methods described in detail in “Handbook of Pharmaceutical Manufacturing Formulations” (Niazi S K, ed, 2004), U.S. Pat. No. 6,111,095; U.S. Pat. No. 6,706,289, U.S. Pat. No. 5,320,840; U.S. Pat. No. 5,446,090; U.S. Pat. No. 5,672,662; U.S. Pat. No. 5,880,255; U.S. Pat. No. 5,942,253; U.S. Pat. No. 6,991,790; U.S. Pat. No. 4,877,608; U.S. Pat. No. 5,032,405; U.S. Pat. No. 5,399,670; U.S. Pat. No. 5,654,403; U.S. Pat. No. 5,730,980; U.S. Pat. No. 5,736,137; U.S. Pat. No. 5,770,700; US Patent Application 20050175708; US Patent Application 20070110775; and Carrasquillo K G et al., 2003, each of which are incorporated in their entirety by reference herein.

### EXAMPLE

The exceedingly hydrophilic nature of the polymer of the conjugate of the present invention can be illustrated by molecular dynamic calculations, as disclosed above (see Detailed Description of the Invention). This methodology is a commonly accepted and widely used method for comparing the physical properties of sets of molecules. The program used for this study is TINKER (TINKER 4.2, distributed by Washington University of St. Louis). The OPLS force field was chosen from the set available in TINKER as the most applicable to the range of synthetic and natural materials examined (Jorgensen, 1996). Periodic boundary conditions were employed with the long range electrostatic forces being calculated by the Ewald summation procedure.

Each polymer model was generated according to the following procedure. The appropriate monomer unit was manually generated from a set of template molecules and functional groups. It was then connected with nine other monomer units to form the corresponding linear polymer. If the head group was not a carboxylic acid, it was modified to be so. The conformation of the polymer chain was set to be either an extended chain (all torsion angles set to 180°) or a helical form (with selected torsion angles of 60°). The polymer was then positioned in the center of a rectangular box with approximate dimensions of 50 Angstrom (Å)×25 A×25 A. The remaining space was filled with water molecules spaced to yield a starting density of 1.0.

The initial model was “relaxed” to a lower energy by computing 40 picoseconds (psec) of molecular dynamics allowing only the water molecules to move. The model used for energy calculations was generated from this relaxed structure by computing 50 psec of dynamics with both the cell and water molecules moving. Energies were calculated for snapshots saved at 1.0 psec intervals and averaged over the last 40 psec of this computation.

Initial chain conformations were based on extensive theoretical and experimental studies of polyethyleneoxide (PEG) and its model monomer 1,2 dimethoxy-ethane. PEG's are remarkably soluble for polyethers and much effort has been expended in determining the reason. The chain conformation in solution seems to be at least partly responsible for this effect. Theoretical calculations have indicated that twisting the carbon-carbon torsion angle from the extended 180° form to the 60° helical form allows a strong hydrogen-bond interaction with a solvent water molecule (Bedrov, 1998). For a dilute solution of dimethoxy-ethane, the percent of molecules with the helical torsion is 83%. Published experimental studies have indicated that a large fraction of the carbon-carbon bonds in PEG are in this helical geometry in aqueous solution (Begum, 1997). Thus, two “frozen” chain conformations were generated for the PEG model, carboxy-decaethyleneoxide to represent the limiting range of hydration energy for PEG: the fully extended form with hydration energy of −9.28 kcal/mol/100 A² and the fully helical form with hydration energy of −13.4 kcal/mol/100 A² (FIG. 1 a,b). Because other polymer chains in this study have different numbers of atoms from carboxy-decaPEG, all hydration energies are normalized against the surface area of the polymer. 100 A² was chosen to yield easily comparable magnitudes. This area normalized hydration energy, SAHE, is further normalized against the SAHE of water itself. The water hydration fraction, WHF, represents how well water hydrates the surface of the polymer molecule compared to water hydrating itself (SAHE of −37.5 kcal/mol/100 A²). All energetic comparisons will be made through this unitless number. The fully extended PEG is 0.247 and the coil form is 0.357 in WHF units. The actual value for the PEG polymer should lie between these two extremes.

In order to determine this value a longer set of 500 psec molecular dynamic simulations was run where the polymer chain, cell, and water molecules were allowed to move. The average WHF observed for three calculations, which included both the fully extended and full coil initial conformers, is 0.358. Thus, short PEG chains exist primarily in the coil form in solution. Experiments on a wide range of PEG monomer concentrations suggest that this value will decrease as the PEG chain length is increased³.

On the other hand, polypropylene oxide is considered a hydrophobic polymer. It is often used with PEG to form block copolymers which usually exhibit micellar properties in aqueous solution. Since it shares the same backbone with PEG, it should exhibit similar conformational energy effects when interacting with water. The WHF (SAHE) values for PPO are 0.232 (−8.71 kcal/mol/100 A²) and 0.346 (−13.0 kcal/mol/100 A²) for the extended and coil conformations, respectively (FIG. 1 c,d). The larger methyl group increases the surface area with a hydrophobic group leading to a three percent lower hydration fraction than that of PEG. The carbon bonded to this methyl group is now a stereoactive carbon. Depending on the mechanism of epoxide ring opening, this backbone carbon could be exclusively in the R stereo-conformation (or S), which is referred to as an isotactic polymer, or it could be a strictly alternating R—S—R—S arrangement, which is a syndiotactic polymer, or it could be a random racemic sequence, an atactic polymer. The values above are for the exclusive R form. Table 2 (below) reveals that the RS or syndiotactic version of PPO interacts with water virtually same as the isotactic form. MD studies on short PPO chains have shown that PPO contains a low fraction of the high hydration energy coil conformer. This leads to PPO being a much more hydrophobic polymer which is insoluble in water at even moderate chain lengths.

On the basis of these calculations any polymer with WHF of about 0.3 or below is insufficiently hydrophilic (i.e. hydrophobic) and any polymer with WHF of about 0.4 is hydrophilic. In the present invention an exceedingly hydrophilic polymer is understood to mean a polymer with WHF of about 0.5 or greater.

Another category of water soluble polymers is based on hydrophilic side chain substituted vinyl polymers. Three members of this group which have been used or proposed for use in pharmaceutical and health care applications such as conjugation to pharmaceutically active agents, are poly-hydroxypropyl-methacrylic amide (hpma), poly-acryloylmorpholine (acm), and poly-vinylpyrrolidone (pvp). Vinyl polymers inherently can exist as isotatic, syndiotactic or atactic chains. Isotactic and syndiotactic model chains were generated for each of these. Generating chain conformations is more difficult with these polymers, as the bulky side chains can easily be constructed in geometries which interact strongly with each other or poorly with water. This is particularly true for the morpholine and pyrrolidone rings. As evident from Table 2 below only one conformation of acm and pvp (acm-iso coil and pvp-iso flat: FIG. 1 e,f) interacted significantly with water. These conformations have significant WHF values, 0.45 (−16.9 kcal/mol/100 A²) and 0.44 (−16.4 kcal/mol/100 A²), respectively. In fact, three of the four hpma conformers also appear to have high WHF values, ranging from about 0.43 (−16.1 kcal/mol/100 A²) to about 0.49 (−18.4 kcal/mol/100 A²). Thus, these vinyl derivatives are about as hydrophilic as PEG, or if they are present in the solution predominantly in the highest HWF conformation, even more so (FIG. 1 g,h).

TABLE 2
Surface Normalized Hydration Energy

| Polymer | SAHE | Dev | WHF | Dev |
|---|---|---|---|---|
| carboxypeg coil | −13.4 | 1.3 | 0.357 | 0.035 |
| carboxypeg flat | −9.28 | 1.2 | 0.247 | 0.031 |
| carboxyppoR coil | −13.0 | 0.9 | 0.346 | 0.023 |
| carboxyppoR flat | −8.71 | 0.7 | 0.232 | 0.018 |
| carboxyppoRS coil | −12.1 | 0.8 | 0.322 | 0.021 |
| carboxyppoRS flat | −7.80 | 0.6 | 0.208 | 0.017 |
| hpma-iso coil | −18.4 | 0.9 | 0.491 | 0.024 |
| hpma-iso flat | −11.7 | 1.2 | 0.306 | 0.033 |
| hpma-syn coil | −16.1 | 0.9 | 0.429 | 0.024 |
| hpma-syn flat | −17.2 | 0.9 | 0.459 | 0.024 |
| acrylmorpholine-iso coil | −16.9 | 0.9 | 0.451 | 0.025 |
| acrylmorpholine-iso flat | −11.6 | 0.7 | 0.310 | 0.018 |
| acrylmorpholine-syn coil | −12.0 | 0.7 | 0.321 | 0.018 |
| acrylmorpholine-syn flat | −11.5 | 0.7 | 0.307 | 0.018 |
| vinylpyrrolidone-iso coil | −11.7 | 0.8 | 0.312 | 0.022 |
| vinylpyrrolidone-iso flat | −16.4 | 1.1 | 0.436 | 0.028 |
| vinylpyrrolidone-syn coil | −12.6 | 0.7 | 0.336 | 0.020 |
| vinylpyrrolidone-syn flat | −11.3 | 1.0 | 0.301 | 0.026 |
| glycolate coil | −15.4 | 1.2 | 0.410 | 0.032 |
| glycolate flat | −14.8 | 1.4 | 0.395 | 0.038 |
| lactateR coil | −12.5 | 1.3 | 0.333 | 0.034 |
| lactateR flat | −13.8 | 1.7 | 0.367 | 0.045 |
| lactateRS coil | −12.8 | 1.2 | 0.342 | 0.031 |
| lactateRS flat | −14.4 | 1.1 | 0.385 | 0.028 |
| β-lactate coil | −13.0 | 1.0 | 0.348 | 0.027 |
| β-lactate flat | −11.5 | 1.3 | 0.306 | 0.034 |
| β-glycerateR coil | −20.0 | 1.5 | 0.534 | 0.039 |
| β-glycerateR flat | −16.9 | 1.4 | 0.449 | 0.037 |
| β-glycerateRS coil | −22.1 | 1.4 | 0.589 | 0.037 |
| β-glycerateRS flat | −20.3 | 1.6 | 0.542 | 0.043 |
| α-glycerateR coil | −21.6 | 1.9 | 0.575 | 0.052 |
| α-glycerateR flat | −20.7 | 1.5 | 0.553 | 0.040 |
| α-glycerateRS coil | −20.8 | 1.5 | 0.554 | 0.040 |
| α-glycerateRS flat | −20.9 | 1.2 | 0.557 | 0.031 |

### REFERENCES

### Books

1. Hermanson, GT “Bioconjugate Techniques”. Academic Press, 1996.
2. “Bioconjugation Protocols: Strategies and Methods” (Niemeyer, ed). CM Humana Press, 2004
3. Lundblad R L “Chemical Reagents for Protein Modification” CRC, 2004
4. Braun D., Cherdron H., Rehahn M., Ritter H., Voit B. “Polymer Synthesis: Theory
and Practice: Fundamentals, Methods, Experiments” 4^{th} edition. Springer, 2002
5. “Handbook of Polymer Reaction Engineering” (Thierry Meyer and Jos Keurentjes, eds) Wiley-VCH; 2005.
6. Methods in Biotechnology: Enzymes in Nonaqueous Solvents (Vulfson, E. N. and Hailing, P. J. and Holland, H. L., eds). Humana Press, 2001
7. “Biomaterial Science: An introduction to materials in medicine” Rather, B D, Hoffman, A S, Schoen F J, Lemons J E (eds), 2^{nd} edition. Elsevier academic press, 2004
8. “Handbook of Pharmaceutical Manufacturing Formulations” Sarfaraz K. Niazi (ed.); C.H.I.P.S. Publications (Weimar, Texas), 2004
9. Block J. and Beale J M “Wilson & Gisvold's Textbook of Organic Medicinal and Pharmaceutical Chemistry” 11^{th} edition. Lippincott Williams & Wilkins, 2003
10. “Peptidomimetics Protocols: Methods in Molecular Medicine” Kazmierski W M (ed), Humana Press, Totowa, N.J., USA, 1998

### Articles

1. Bedrov O. Borodin G D, Smith J. Phys. Chem. B102, 5683 (1998)
2. Begum R & Matsuura H. J. Chem. Soc, Faraday Trans. 93, 3839 (1997)
3. Blake et al. Methods Mol. Biol. 356, 367-377 (2007)
4. Carrasquillo K G et al. Invest Opthalmol. Vis. Sci., 44:290-9 (2003)
5. Chen T & Feng X. Assay Drug Dev Technol. 4, 473-82 (2006)
6. Douris V et al. Adv Virus Res., 68, 113-156 (2006)
7. Eglen R M. Comb Chem High Throughput Screen., 8, 311-8 (2005)
8. Hamdan F F et al. J. Biomol. Screen. 10, 463-475 (2005)
9. Hoste et al. 2004 International Journal of Pharmaceutics 277 119-131 (2004)
10. Jorgensen W L, Maxwell D S, Tirado-Rives J J. JACS, 118, 11225 (1996)
11. Jose J. Appl Microbiol Biotechnol. 69, 607-614 (2006)
12. Krausz E. Mol. Biosyst. 3, 232-240 (2007)
13. Leifert W R et al. J. Biomol. Screen. 10, 765-779 (2005)
14. Luesch H. Mol. Biosyst. 2, 609-620 (2006)
15. Lundholt B K et al. Assay Drug Dev Technol. 4, 679-688 (2006)
16. Perrimon N & Mathey-Prevot B. Genetics, 175, 7-16 (2007)
17. Proll G et al. J. Chromatogr A., 1161, 2-8 (2007)
18. Rausch O. IDrugs 8:573-577 (2005)
19. Rausch O. Curr Opin Chem Biol. 10, 316-320 (2006)
20. Trask O J et al. Methods Enzymol. 414, 419-439 (2006)
21. Veronese F M. Biomaterials 22 405-417 (2001)
22. Vogt A et al. Oncol Res. 14, 305-314 (2004)
23. Zanella F et al. Assay Drug Dev Technol. 5, 333-341 (2007)

### US Patents and US Patent Applications

1. U.S. Pat. No. 4,877,608
2. U.S. Pat. No. 5,032,405
3. U.S. Pat. No. 5,320,840
4. U.S. Pat. No. 5,399,670
5. U.S. Pat. No. 5,446,090
6. U.S. Pat. No. 5,654,403
7. U.S. Pat. No. 5,672,662
8. U.S. Pat. No. 5,730,980
9. U.S. Pat. No. 5,736,137
10. U.S. Pat. No. 5,770,700
11. U.S. Pat. No. 5,880,255
12. U.S. Pat. No. 5,942,253
13. U.S. Pat. No. 6,111,095
14. U.S. Pat. No. 6,387,617
15. U.S. Pat. No. 6,461,603
16. U.S. Pat. No. 6,653,458
17. U.S. Pat. No. 6,706,289
18. U.S. Pat. No. 6,852,535
19. U.S. Pat. No. 6,991,790
20. U.S. Pat. No. 7,037,894
21. U.S. Pat. No. 7,214,776,
22. US Patent Application 20050175708
23. US Patent Application 20070110775
24. US Patent Application 20060182692

The invention claimed and described herein is not to be limited in scope by the specific embodiments herein disclosed since these embodiments are intended as illustrations of several aspects of the invention. Indeed, various modifications of the invention in addition to those shown and described herein will become apparent to those skilled in the art from the foregoing description. Such modifications are also intended to fall within the scope of the appended claims.

A number of references are cited herein, the entire disclosures of which are incorporated herein, in their entirety, by reference.

Contents

| | |
|---|---|
| CONJUGATES OF POLYMER AND PHARMACOLOGICALLY | 1 |
| ACTIVE AGENTS AND A NOVEL POLYMER BLEND | |
| Background of the Invention | 1 |
| Field of Invention | 1 |
| Description of the Related Art | 1 |
| Summary of the Invention | 7 |
| Brief Description of Drawings | 9 |
| Detailed Description of the Invention | 9 |
| Polymers of the Invention | 10 |
| Synthesis of Polymers of the Invention | 16 |
| Assaying Polymers of the Invention | 17 |
| Pharmacologically Active Agents of the Invention | 18 |
| Oligo- and Polypeptides and Peptidomimetics of the Invention | 19 |
| Small Molecule Drugs | 20 |
| Nucleic Acid Polymers of the Invention | 21 |
| Conjugates of the Invention | 22 |
| Attachment of Polymers | 23 |
| Polymer Blends of the Invention | 25 |
| Formulation of Conjugates of the Invention | 26 |
| Example | 27 |
| References | 33 |
| Books | 33 |
| Articles | 34 |
| US Patents and US Patent Applications | 35 |
| Claims | 38 |
| Abstract | 42 |

1: A conjugate of a polymer and a pharmacologically active agent, wherein the polymer is (a) non-proteinaceous, (b) synthetic, (c) biodegradable and (d) exceedingly hydrophilic.

2: The conjugate of claim 1, wherein the polymer contains between 0.2 and 1.8 mole of hydroxyl groups per mole of monomer.

3: The conjugate of claim 1, wherein the polymer is a linear or substantially linear polymer.

4: The conjugate of claim 1, wherein the polymer is a branched or dendrimeric polymer.

5: The conjugate of claim 1, wherein the polymer is a polymer or co-polymer of glyceric acid or derivatives, homologs, or analogs thereof.

6: The conjugate of claim 1, wherein the monomers are single enantiomers.

7: The conjugate of claim 1, wherein one or more polymers are attached to the pharmaceutically active agent.

8: The conjugate of claim 1, wherein one or more pharmaceutically active agents are attached to the polymer.

9: The conjugate of claim 1, wherein the pharmaceutically active agent is selected from the group consisting of small molecules, oligopeptides, oligopeptide derivatives, oligopeptide analogs, polypeptides, polypeptide derivatives, polypeptide analogs, proteins, protein complexes, antibodies, peptidemimetics, aptamers, and RNAi molecules.

10: The conjugate of claim 1, wherein the polymer and the pharmaceutically active agent are coupled through a linker.

11. The conjugate of claim 1, wherein the polymer is a polymer or co-polymer of glyceric acid or derivatives, homologs, or analogs thereof and covalently linked or attached to another polymer, and wherein the resulting linked polymers are substantially synthetic, biodegradable, non-proteinaceous and exceedingly hydrophilic in combination.

12: A method for the preparation of polymers conjugated with pharmaceutically active agents comprising:
(1) synthesis of a non-proteinaceous, biodegradable, and exceedingly hydrophilic polymer;
(2) obtaining the pharmaceutically active agent;
(3) covalently coupling the polymer to the pharmaceutically active agent; and
(4) purifying the resulting pharmaceutically active conjugate

13: The method of claim 12, whereby the polymer that is synthesized is a polymer wherein the polymer is a polymer or co-polymer of glyceric acid or derivatives, homologs, or analogs thereof, and whereby the polymer or copolymer has an average molecular weight between 1 kD and 100 kD.

14: The method of claim 12, wherein the biodegradable polymer or co-polymer is prepared with the aid of biological catalysts.

15: The method of claim 12, wherein the pharmacologically active agent is selected from the group consisting of small molecules, oligopeptides, oligopeptide derivatives, oligopeptide analogs, polypeptides, polypeptide derivatives, polypeptide analogs, proteins, protein complexes, antibodies, peptidemimetics, aptamers, and RNAi molecules.

16: A blend of at least two polymers, wherein:
(a) the first polymer is a synthetic, non-proteinaceous, biodegradable, and exceedingly hydrophilic polymer;
(b) the other, or at least one of the other, polymers in the blend is biodegradable to a degree comparable to that of the first polymer; and
(c) the mass ratio of the first polymer to the combined mass of the rest of the polymers in the blend is between 97:3 to 3:97

17: The blend of claim 16, wherein the first polymer is a polymer or co-polymer of glyceric acid or derivatives, homologs, or analogs thereof.

18: The blend of claim 16, wherein the first polymer is linear, substantially linear, branched, or dendrimeric.

19: The blend of claim 16, wherein the monomers of the first polymer are single enantiomers.

20: Use of the blend of claim 16 in pharmaceutical and health care products and applications.
